(19) United States
(12) Patent Application Publication
(10) Pub. No.: US 2004/0009182 A1
(43) Pub. Date: Jan. 15, 2004

(54) Method and compositions using anthrax immune globulin to provide passive immunity against lethal infections from bacillus anthracis

(76) Inventors: Robert C. Myers, Perrinton, MI (US); Arthur Thomas Waytes, Dewitt, MI (US)

Correspondence Address: FOSTER, SWIFT, COLLINS & SMITH, P.C., 313 SOUTH WASHINGTON SQUARE, LANSING, MI, 48933, US

(21) Appl. No.: 10/402,624

(22) Filed: Mar. 28, 2003

Related U.S. Application Data
(60) Provisional application No. 60/369,123, filed on Apr. 1, 2002.

(51) Int. Cl. A61K 39/40; A61K 39/00; A61K 39/38; A61K 6/00; A61K 7/00

(52) U.S. Cl. 424/184.1

(57) ABSTRACT

Methods and compositions capable of quickly transferring antibody-mediated protection against lethal infections of B. anthracis in an animal without benefit of vaccination against B. anthracis. The present invention method includes providing plasma from donors, said plasma having a measurable level of immunologically active immunoglobulin against anthrax; and administering a predetermined quantity of said plasma product to the animal, wherein an antibody-mediated protection against lethal infections of B. anthracis is elicited. Methods of manufacturing a composition to transfer passive anthrax immunity to an animal include providing plasma from hyper-immunized donors, having a measurable level of immunologically active immunoglobulin against anthrax; and purifying said plasma that substantially preserves the titer of the immunoglobulin in the plasma. The plasma may be screened for infectious diseases and for toxin neutralization antibodies (TNA). The invention may also include the steps of pooling the plasma from donors and inactivating residual viral activity.

This application claims priority to a U.S. Provisional Application Serial No. 60/369,123 titled, “Method and System Using Anthrax Immune Globulin to Provide Passive Immunity Against Anthrax,” filed Apr. 1, 2002. The entire disclosure of Ser. No. 60/369,123 is incorporated hereby by reference in its entirety.

### FIELD OF THE INVENTION

The present invention generally relates to methods and compositions to transfer passive immunity, and specifically to methods and compositions to transfer passive immunity against lethal infections of Bacillus anthracis (B. anthracis) using an immunoglobulin, and methods of manufacture.

### BACKGROUND OF INVENTION

Anthrax is a well-known disease and was one of the first diseases to be described in association with its causative organism, Bacillus anthracis (B. anthracis) (1). [It is noted that this numeral reference, and others that similarly follow, references a correspondingly numbered citation in the Literature Cited section, infra.] Although well characterized as a disease, it is only in recent years that the molecular basis of anthrax has begun to be understood. The principal virulence factor of B. anthracis is a multi-component toxin secreted by the organism, consisting of three separate gene products designated protective antigen (PA), lethal factor (LF) and edema factor (EF).

The genes encoding the B. anthracis toxin components PA, LF, and EF (pagA, lef, and cya, respectively) are located on a 184-kb plasmid designated pX01 carried by all strains of B. anthracis (2). PA (735 aa, Mr 82,684) is a single chain protein that binds to anthrax toxin receptor (ATR) (3) on the cell surface and subsequently undergoes a furin-mediated cleavage to yield a 63-kDa receptor-bound product (4, 5, 6, 7). The 63-kDa PA fragment forms a heptameric complex on the cell surface that is capable of interacting with either the 90-kDa LF protein or the 89-kDa EF protein, which are subsequently internalized (8, 7). LF (776 aa, Mr 90,237) is a zinc metalloprotease that cleaves several isoforms of MAP kinase kinase (Mekl, Mek2, Mek3), thereby disrupting signal transduction events within the cell and eventually leading to cell death (9, 10). The EF protein (767 aa, Mr 88,808) is a calmodulin-dependent adenylate cyclase that causes deregulation of cellular physiology, leading to clinical manifestations that include edema (11). The LF protein combines with PA to form what is referred to as lethal toxin (Letx), which is considered the primary factor responsible for the lethal outcome of anthrax infection.

The most effective method for preventing lethal anthrax infections known in the art is vaccination. One of the earliest successful vaccines was an attenuated strain of B. anthracis used by Louis Pasteur to vaccinate sheep against anthrax (12). The current human FDA-approved anthrax vaccine in the U.S. (BIOTHRAX, BioPort Corporation, Lansing, Mich.) is produced from the culture supernatant fraction of the V770-NP1-R strain of B. anthracis and consists principally of the PA antigen adsorbed onto aluminum hydroxide. Protection against anthrax infection is associated with a humoral immune response directed against PA (13, 14). Some evidence suggests that EF and LF may also contribute to specific immunity (15, 16), although these components have not been formulated into a subunit vaccine.

Antibiotic therapy may also be used to prevent the lethal effects from an infection of B. anthracis. Unfortunately, antibiotic therapy used in post anthrax exposure animals without benefit of prior vaccination, does not have any direct effect on the toxins secreted by B. anthracis. In addition, strains of B. anthracis have been developed that are resistant to certain antibiotics, including ciprofloxacin (17). If infection with such a resistant organism occurred, anthrax immunoglobulin may provide protection. The protective effect of antibodies directed against PA, as well as against LF and other vaccine components in a recipient of, for example BIOTHRAX, anthrax vaccine takes several weeks to develop. A quicker and more robust response follows booster immunizations.

Once developed, this antibody-mediated protection may be expected to be immediately transferable to a second individual. If given in adequate quantity, it would be expected to neutralize toxins produced during an ongoing B. anthracis infection, and may have activity against the bacteria themselves. Such passive protection has been demonstrated for other diseases such as rabies, tetanus, hepatitis B, vaccinia, varicella-zoster, cytomegalovirus, and respiratory syncytial virus. The passive protection occurs with antibody preparations derived from animal hosts immunized with the respective vaccines.

In a recent study (18), passive anthrax immunity was conferred to guinea pigs using hyperimmune serum obtained two weeks after a 3-dose regimen from immunized guinea pigs. Doses of hyperimmune serum with corresponding titers of 2,400 to 24,000 were injected intramuscularly into naive guinea pigs. One day after serum transfer, the guinea pigs were challenged by intradermal injection of 2000 spores (40 LD₅₀s) from the B. anthracis Vollum strain. Guinea pigs with toxin neutralizing antibody (TNA) titers greater than 220 were fully protected. Results from passive immunization correlated well with direct immunization of the guinea pigs followed by anthrax challenge, wherein, full protection was observed when circulating TNA titers were 300 or greater (18).

The ability of anthrax vaccine to elicit an immune response in humans is also well-documented (19, 20, 21, 22). Unfortunately, these studies do not have a standardized method of antibody measurement. This makes it difficult to compare human responses among studies. In the above referenced guinea pig study, the TNA assay correlated much better with extent of protection than did the anti-PA assay (18). TNA titers resulting from direct inoculation of the guinea pigs also correlated well with the circulating TNA titers after passive immunization.

While there is a desire and a need to provide passive anthrax immunity in humans, no such method or system is known in the art. TNA titers in humans after direct immunization may be predictive of circulating TNA titers targeted for post passive immunization with anthrax immune globulin. Unfortunately, this is unknown in the art.

Thus there is a desire and a need to provide methods and compositions capable of immediate transfer of antibody-mediated protection against lethal infections of B. anthracis in an animal host without benefit of vaccination against B. anthracis.

### SUMMARY OF THE INVENTION

Accordingly, the present invention provides methods and compositions capable of quickly transferring antibody-mediated protection against lethal infections of B. anthracis in an animal without benefit of vaccination against B. anthracis.

The present invention is a method for transferring passive anthrax immunity to an animal, including providing plasma from donors, said plasma having a measurable level of immunologically active immunoglobulin against anthrax; and administering a predetermined quantity of said plasma product to the animal, wherein an antibody-mediated protection against lethal infections of B. anthracis is elicited.

The present invention includes a method of manufacturing a composition to transfer passive anthrax immunity to an animal, including providing plasma from donors, said plasma having a measurable level of immunologically active immunoglobulin against anthrax; and purifying said plasma that substantially preserves the titer of the immunoglobulin in the plasma. The invention can also include hyper-immunizing said donors using an anthrax immunogenic composition; collecting plasma from said donors at a predetermined interval, twice weekly during the two to three months following immunization; and screening the plasma for infectious diseases and for toxin neutralization antibodies (TNA). TNA's may be screened using a direct enzyme-linked immuno-sorbent assay (ELISA) or an in vitro cytotoxicity method. The invention may also include the steps of pooling the plasma from donors and. inactivating residual viral activity.

The present invention also includes a composition of antibody preparation having a measurable level of immunologically active immunoglobulin against anthrax, such as anthrax (immunoglobin G) IGG or other TNA.

Other features of the present invention will become more apparent to persons having ordinary skill in the art to which the present invention pertains from the following description and claims taken in conjunction with the accompanying figures.

### DETAILED DESCRIPTION OF THE INVENTION

The present invention relates to methods and compositions capable of quickly transferring antibody-mediated protection against lethal infections of B. anthracis in an animal, including humans, without benefit of vaccination against B. anthracis, including methods of manufacture.

In cases where a B. anthracis infection occurs with an antibody resistant organism, an anthrax immunoglobulin of the present invention composition and method may provide protection against the potentially lethal effects. The protective effect of antibodies directed against PA, as well as against LF and other immunogenic composition components in a recipient of prior art anthrax immunogenic compositions takes several weeks to develop. A quicker and more robust response follows booster immunizations.

Once developed, this antibody-mediated protection may immediately be transferable to a different animal host. If given in adequate quantity, it would be expected to neutralize toxins produced during an ongoing B. anthracis infection, and act against the bacteria itself. Passive protection occurs with antibody preparations derived from individuals immunized with the respective immunogenic compositions.

As known in the art, passive anthrax immunity through the development of toxin neutralizing antibodies (TNAs) is conferred to guinea pigs using hyperimmune serum obtained two weeks after a 3-dose regimen from immunized guinea pigs. Guinea pigs with TNA titers greater than 220 were fully protected. Results from passive immunization correlated well with direct immunization of the guinea pigs followed by anthrax challenge, wherein, full protection was observed when circulating TNA titers were 300 or greater(18).

The ability of anthrax immunogenic composition to elicit an immune response in humans is well documented (19, 20, 21, 22). TNA titers in humans after direct immunization may be predictive of circulating TNA titers targeted for post passive immunization with anthrax immune globulin. Unfortunately, this is unknown in the art.

Accordingly, the present invention is a method, composition, and method of manufacture to provide passive anthrax immunity in humans for prevention and/or treatment of anthrax disease. Many systems and methods of developing such immunity are possible using the features and advantages of the present invention. Nevertheless, the following is provided as an illustration of but one such possibility.

One embodiment of the present invention begins with immunizing and/or hyper-immunizing plasma donors. This can be done using BioPort's (Lansing, Mich.) BIOTHRAX anthrax immunogenic composition under Investigational New Drug Application as required by FDA. The resultant plasma from the donors has a level of immunologically active immunoglobulin.

Plasma from donors may then be collected at predetermined intervals. Such intervals can be those governed by 21 Code of Federal Regulations (CFR) using state of the art plasmapheresis equipment and facilities, in compliance with all FDA requirements. For illustration purposes only, such an interval could be twice weekly during the two to three months following immunization.

Next, the plasma may optionally be screened. This can be in a licensed laboratory using FDA cleared tests for known infectious diseases as required by 21 (CFR). The screening could also include screening for anti-PA and/or toxin neutralization antibodies (TNA). This can be accomplished using a direct enzyme-linked immuno-sorbent assay (ELISA) and/or by an in vitro cytotoxicity method. Other methods may be fully validated using GMP standards for test method validation.

Next, plasma from seroconverted donors may be pooled followed by purifying immune globulins. The purifying step may be accomplished via a Modified Cohn Method 6 (23) known in the art. The Modified Oncley Method 9 (24) may be used to purify the immune globulin G (IGG) from the initial crude preparation. The immune globulin may be further purified for intravenous use.

Manufacture of the immune globulin may also involve a standard solvent detergent step, known in the art, for inactivation of residual viral activity in the composition or a similarly robust viral inactivation step.

It will be clear that the present invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and examples is hereby incorporated herein by reference.

We claim:

1. A method for transferring passive anthrax immunity to an animal, comprising the steps of:
providing plasma from donors, said plasma having a measurable level of immunologically active immunoglobulin against anthrax; and
administering a predetermined quantity of said plasma product to the animal, wherein an antibody-mediated protection against lethal infections of B. anthracis is elicited.

2. A method of manufacturing a composition to transfer passive anthrax immunity to an animal, the method comprising the steps of:
providing plasma from donors, said plasma having a measurable level of immunologically active immunoglobulin against anthrax; and
purifying said plasma that substantially preserves the titer of the immunoglobulin in the plasma.

3. The method of claim 2 further comprising the steps of:
hyper-immunizing said donors using an anthrax immunogenic composition; and
collecting plasma from said donors at a predetermined interval.

4. The method of claim 3, wherein the predetermined interval is twice weekly during the two to three months following immunization.

5. The method of claim 2, further comprising the step of screening the plasma.

6. The method of claim 5, wherein the step of screening comprises screening for infectious diseases.

7. The method of claim 5, wherein the step of screening comprises screening for toxin neutralization antibodies (TNA).

8. The method of claim 7, wherein the step of screening for TNA comprises screening using a direct enzyme-linked immuno-sorbent assay (ELISA).

9. The method of claim 7, wherein the step of screening comprises screening using an in vitro cytotoxicity method.

10. The method of claim 2, further comprising the step of pooling the plasma from donors.

11. The method of claim 2, further comprising the step of inactivating residual viral activity.

12. The method of claim 2, wherein the step of purifying said plasma uses a Modified Cohn Method.

13. A composition of antibody preparation comprising a measurable level of immunologically active immunoglobulin against anthrax.

14. The composition of claim 13, wherein the immunoglobin is anthrax IGG.

15. The composition of claim 13, wherein the immunoglobin is toxin neutralization antibodies.

16. The composition of claim 13, further comprising donor plasma.

17. The composition of claim 16, wherein the plasma is screened.

18. The composition of claim 16, wherein the plasma is screened for infectious diseases.

19. The composition of claim 16, wherein the plasma is screened for toxin neutralization antibodies.

20. The composition of claim 16, wherein the plasma is purified.
